(19) United States
(12) Patent Application Publication
(10) Pub. No.: US 2022/0296348 A1
(43) Pub. Date: Sep. 22, 2022

(54) Horse Bandage

(71) Applicant: Anne-Marie Klose, Hamburg (DE)

(72) Inventor: Anne-Marie Klose, Hamburg (DE)

(21) Appl. No.: 17/202,659

(22) Filed: Mar. 16, 2021

(51) Int. Cl. A61D 9/00; A61F 13/62

(52) U.S. Cl. CPC A61D 9/00; A61F 13/622

(57) ABSTRACT

A horse bandage having a winding section (2) for wrapping around a horse's leg and a closure strip (3) with a piece of hook-and-loop fastener (9) arranged on the outside of the bandage and spaced apart from an inner end of the winding section (6) facing the closure strip (3), and a counterpart piece of hook-and-loop fastener (7) arranged on the inside of the bandage at the inner end of the winding section (6), and a further piece of hook-and-loop fastener (11) arranged on the outside of the bandage at an inner end of the closure strip (4), and a further counterpart piece of hook-and-loop fastener (12) arranged at an outer end of the closure strip (8), wherein the inner end of the closure strip (4) is fastened to the winding section (2) on the outside of the bandage and at the end of the piece of hook-and-loop fastener (9).

### BACKGROUND OF THE INVENTION

### Field of the Invention

The invention relates to a horse bandage with a winding section for wrapping around a horse's leg.

### Description of the Related Art

Naturally, horse bandages are well known in the prior art. For protection of the horse's leg a horse bandage is wrapped around the lower region of the horse's leg.

In particular the horse's foreleg is at risk when jumping, and for protection the horse bandage is wrapped around it. Furthermore, the horse bandage also serves for protection against injuries by the hind leg.

A disadvantage of the known horse bandages is that in particular after the wrapping of the horse's leg a closure is clearly recognisable and it is hardly possible to achieve a visually uniform appearance when all four of the horse's legs are wrapped. Furthermore, the closure of a horse bandage with an individual hook-and-loop fastener is insecure.

### SUMMARY OF THE INVENTION

An object of the present invention is to improve a horse bandage mentioned in the introduction and to reduce the above-mentioned disadvantages as much as possible.

This object is achieved by a horse bandage as mentioned above with the features of claim 1.

The horse bandage has a winding section for wrapping a horse's leg and a closure strip.

The horse bandage has a piece of hook-and-loop fastener arranged on the outside of the bandage and spaced 10 cm to 50 cm apart from an inner end of the winding section facing the closure strip, and a counterpart piece of hook-and-loop fastener arranged on the inside of the bandage at the inner end of the winding section. The piece of hook-and-loop fastener and the counterpart piece of hook-and-loop fastener together form a hook-and-loop fastener.

According to the invention a further piece of hook-and-loop fastener is arranged on the outside of the bandage at an inner end of the closure strip, and a further counterpart piece of hook-and-loop fastener is arranged on an outer end of the closure strip, and the inner end of the closure strip is fastened to the winding section, specifically at the end of the piece of hook-and-loop fastener, i.e. the inner end of the closure strip is fastened, preferably sewn, between the piece of hook-and-loop fastener and the inner end of the winding section or directly to the inner winding end of the section. Thus the closure strip may also be a direct extension of the winding section. In another embodiment, at the same time the closure strip is fastened as an additional flap to the winding section on the outside of the bandage.

After the wrapping of the horse's leg with the winding section of the horse bandage the piece of hook-and-loop fastener and the counterpart piece of hook-and-loop fastener are connected to one another and form the hook-and-loop fastener. For this purpose the piece of hook-and-loop fastener and the counterpart piece of hook-and-loop fastener are arranged on the outside of the bandage or on the inside of the bandage on winding section and are spaced apart from one another by 10 cm to 50 cm. This corresponds approximately to the possible winding circumferences.

Advantageously both the piece of hook-and-loop fastener and also the counterpart piece of hook-and-loop fastener or at least one of the two pieces has a dimension in the longitudinal direction which takes account of the tolerances and differences in the winding circumferences. Advantageously both the piece of hook-and-loop fastener and also the counterpart piece of hook-and-loop fastener are each approximately 20 cm long, preferably 10 to 30 cm long.

According to the invention act the further piece of hook-and-loop fastener and the further counterpart piece of hook-and-loop fastener additionally interact. They form the further hook-and-loop fastener. Also they are preferably spaced apart from one another in the longitudinal direction by between 10 and 50 cm, preferably between 20 and 40 cm. They can likewise have lengths in each case of preferably 5 to 10 cm.

Advantageously the closure strip is consistent along its longitudinal direction. In other words its width is preferably constant along its entire longitudinal extent, and preferably the impression on the outside of the bandage is also constant or at least periodic in the longitudinal direction, so that when the horse's leg is wrapped with the closure strip the same visual impression is produced round the circumference. Due to this external configuration of the closure strip, the location on the horse's leg at which the further piece of hook-and-loop fastener and the further counterpart piece of hook-and-loop fastener are brought into contact with one another is unimportant for the visual impression.

Particularly preferably, the closure strip is narrower than the winding section, so that the closure strip with the further hook-and-loop fastener additionally fixes the winding section on the leg and forms the visual impression of a circumferential narrow strip.

Particularly preferably the closure strip is formed resiliently, so that the closure strip also exerts additional pressure on the horse bandage and due to its resilience it can additionally be adapted to different circumferential lengths.

### BRIEF DESCRIPTION OF THE DRAWINGS

The invention is described with reference to an embodiment in three drawings. In the drawings:

FIG. 1 shows a closure section of a bandage according to the invention in a view of the inside of the bandage.

FIG. 2 shows the closure section in FIG. 1 in a view of the outside of the bandage,

FIG. 3 shows a side view of a horse bandage according to the invention.

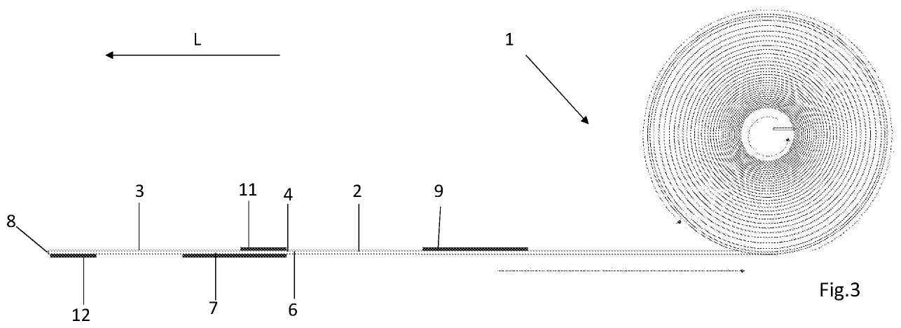

### DETAILED DESCRIPTION OF THE SEVERAL EMBODIMENTS

A horse bandage 1 comprises a winding section 2 and a closure strip 3. The winding section 2 is produced from fleece or wool. The winding section 2 can have a length of 3 m to 4 m and a width of 10 cm to 15 cm.

The closure strip 3 is produced from a resilient material. An inner end 4 of the closure strip 3 is sewn to an inner end 6 of the winding section 2. The winding section 2 has a length of 35 cm and a width of 5 cm. The closure strip 2 is formed resiliently.

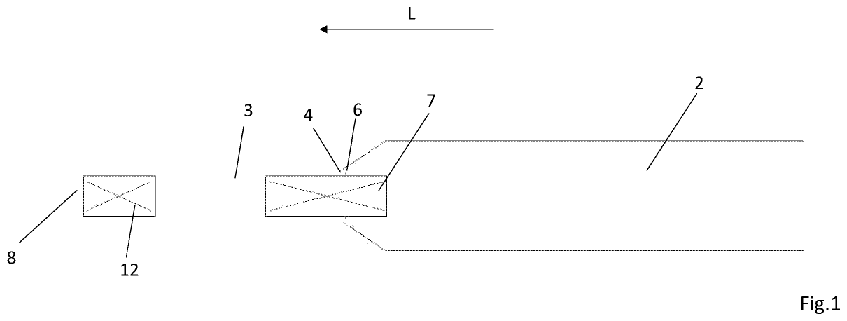

According to FIG. 1 a counterpart piece of hook-and-loop fastener in the form of a hook strip 7, which has a length of 20 cm and a width of 5 cm, is sewn to the inside of the bandage at the inner end of the winding section 6. A piece extends some way into the winding section 2 and also some way on the inside of the bandage on the closure strip 3 in the direction of an outer end of the closure strip 8. The hook strip 7 interacts with a loop strip 9. The loop strip 9 is sewn to the winding section on the outside of the bandage 2. The winding section 9 has a length of 20 cm and a width of 5 cm. Centre points of the hook strip 7 and of the loop strip 9 are spaced apart from one another by approximately 30 cm in a longitudinal direction L of the winding section 2. The spacing corresponds approximately to the winding circumference of a horse's leg, wherein winding circumference means the length required for winding around the horse's leg, wherein a bandage underlay is optionally also laid directly onto the horse's leg.

The closure strip 3 is provided for additional fastening of the horse bandage 1. A further hook strip 11 on the outside of the bandage with a length of 5 cm and a width of 5 cm and a further loop strip 12 on the inside of the bandage with a length of 5 cm and a width of 5 cm are sewn to the closure strip 3, and are likewise sewn with their centre points spaced apart from one another by approximately 30 cm onto the opposing sides on the closure strip 3. The further hook strip 11 is sewn directly at the inner end of the closure strip 4, and the further loop strip 12 is sewn directly to the outer end of the closure strip 8. The closure strip 3 is preferably consistent in the longitudinal direction L, in particular visually consistent, so that with a single wrapping of the winding section 2 on the horse's leg and closing of the closure strip 3 a circumferentially uniform impression of the horse bandage 1 is produced for the observer.

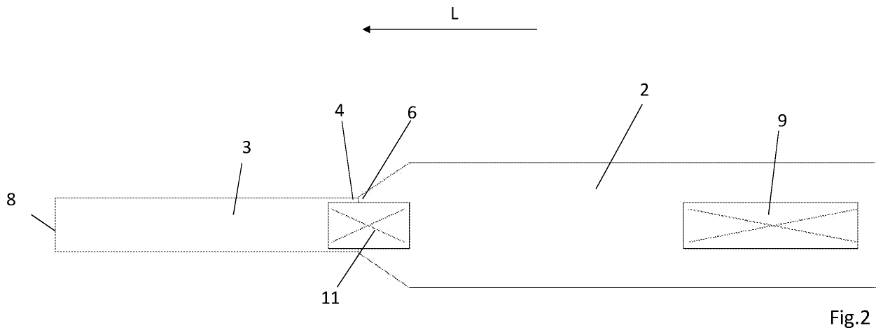

The horse bandage 1 is wound conventionally, by winding it with the inside of the bandage directly onto the horse's leg or onto a bandage underlay resting directly on the horse's leg, and the horse's leg is wrapped as much as required until only the closure section of the horse bandage 1 shown in FIGS. 1 and 2 is exposed. The winding section 2 is then laid once around the horse's leg, and the loop strip 9 and the hook strip 7 are connected to one another. Then the closure strip 3 is wound precisely once around the horse's leg, and the further loop strip 12 and the further hook strip 11 are connected to one another. Thus a doubly secured and aesthetically pleasing bandaging of the horse's leg and in particular of all four of the horse's legs is produced.

### LIST OF REFERENCES

1 horse bandage
2 winding section
3 closure strip
4 inner end of the closure strip
6 inner end of the winding section
7 hook strip
8 outer end of the closure strip
9 loop strip
11 further hook strip on the outside of the bandage
12 further loop strip on the inside of the bandage
L longitudinal direction

What is claimed is:

1. A horse bandage comprising: a winding section (2) for wrapping around a horse's leg and a closure strip (3) with a piece of hook-and-loop fastener (9) arranged on the outside of the bandage and spaced 10 cm to 50 cm apart from an inner end of the winding section (6) facing the closure strip (3), and a counterpart piece of hook-and-loop fastener (7) arranged on the inside of the bandage at the inner end of the winding section (6), and a further piece of hook-and-loop fastener (11) arranged on the outside of the bandage at an inner end of the closure strip (4), and a further counterpart piece of hook-and-loop fastener (12) arranged at an outer end of the closure strip (8), wherein the inner end of the closure strip (4) is fastened to the winding section (2) on the outside of the bandage and at the end of the piece of hook-and-loop fastener (9).

2. The horse bandage according to claim 1, wherein the closure strip (3) is sewn to the inner end of the closure strip (4) directly at the inner end of the winding section (6).

3. The horse bandage according to claim 1, wherein a spacing between the piece of hook-and-loop fastener (9) and the counterpart piece of hook-and-loop fastener (7) and between the further piece of hook-and-loop fastener (11) and the further counterpart piece of hook-and-loop fastener (12) is in each case 10 to 50 cm, preferably 20 to 40 cm.

4. The horse bandage according to claim 1, wherein the closure strip (3) is consistent along its longitudinal direction (L).

5. The horse bandage according to claim 1, wherein the closure strip (3) is narrower than the winding section (2).

6. The horse bandage according to claim 1, wherein the closure strip (3) is resilient.
